(19) United States
(12) Patent Application Publication
(10) Pub. No.: US 2007/0042007 A1
(43) Pub. Date: Feb. 22, 2007

(54) Topical composition for delivery of salicylate esters

(76) Inventors: Joseph Schwarz, Richmond Hill (CA); Michael Weisspapir, Toronto (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/206,858

(22) Filed: Aug. 19, 2005

(51) Int. Cl. A61K 31/60; A61K 9/00

(52) U.S. Cl. 424/400; 514/159; 514/165

(57) ABSTRACT

The present invention provides a self-emulsifiable composition and a stable topical emulsion comprising same, which comprises salicylate esters, oils and surfactants and which does not require organic solvents. The invention also provides methods for manufacturing the compositions. The topical compositions of the invention can be used to enhance delivery of salicylate esters and are useful in the prevention and treatment of pain and inflammation.

### FIELD OF THE INVENTION

The present invention relates to a topical composition for the delivery of salicylate esters, processes for making same and methods and uses related thereto.

### BACKGROUND OF THE INVENTION

Multiple types of topical applications in the form of lotions, creams, ointments, rubs, liniments, balms and solutions, directed to the relief of arthritic pain, muscle soreness, neck tenderness, backaches, tendinitis, and bursitis are well-known. Such formulations typically include combinations of anti-inflammatory drugs or other painkillers, counterirritants, and organic solvents, with essential oils as penetration enhancers. The efficacy of these medications in pain management is still not adequate. U.S. Pat. No. 6,528,076 entitled “Topical compositions and methods for treating pain” describes topical compositions for treating pain, and methods of using same, wherein the compositions comprise an effective amount of acetone, emollient and a salicylate-based compound.

U.S. Pat. No. 6,060,062 entitled “Liquid composition for the topical application to relieve arthritic pain”, discloses a liquid liniment composition directed at relief of arthritic pain comprising banana peel extracts, alcohol, parsley, turpentine and either acetylsalicylic acid or wintergreen isopropyl alcohol (which contains methylsalicylate).

U.S. Pat. No. 4,353,896 entitled “Penetrating topical medicament” describes a topical medicament used in treating athletic injuries and subdermal pain comprising an analgesic (such as methyl salicylate), one or more emulsifiers, penetrating solvent (dimethylsulfoxide (DMSO)) and an alcoholic carrier. DMSO is a penetrating carrier and has been shown to deliver active compounds through the skin, however, questions remain as to the safety of DMSO for human use, and several side effects in humans are known.

The formulations disclosed in these patents all have limitations and are not ideal. For instance, the patents above all utilize organic solvents, such as aprotic solvents, with associated toxicity (such as acetone or DMSO). Also limitations exist with regard to penetration potential for transdermal salicylate delivery.

Submicron emulsions show high penetration potential for transdermal delivery of incorporated drugs, such as disclosed in U.S. Pat. No. 6,113,921, entitled “Topical and transdermal delivery system utilizing submicron oil spheres”. However, the method of preparing the emulsion disclosed therein is unsuitable for preparation of emulsions with high levels of oil phases. Furthermore, the manufacture of submicron emulsions by this method requires sophisticated equipment, such as high pressure homogenizers or microfluidizers. The application of extensive force and energy during the homogenization process causes degradation by hydrolysis or oxidation of sensitive active ingredients, such as salicylates. Additionally, since the anti-inflammatory activity of salicylates is relatively low, a high content of the active component is required. However, a high concentration of salicylates has been associated with destabilization of drug-loaded emulsions.

U.S. Pat. No. 5,965,160 describes a self-emulsifiable composition for oral drug delivery. The system contains hydrophobic long-chain aliphatic amines having distinct toxicity and thus can be used only in limited cases.

As such, there exists a need for a topical salicylate medication that overcomes the limitations of the prior art and that has improved efficacy.

### SUMMARY OF THE INVENTION

In one embodiment, the invention provides a self-emulsifiable composition comprising a salicylate ester, oils and non-ionic surfactants. In one embodiment, the oils comprise a combination of alphatic and aromatic oily excipients.

In one embodiment, the salicylate ester comprises 20-50% by weight of the self-emulsifiable composition, providing about 5-25% by weight content of salicylate in the formed emulsion after dilution of the self-emulsifiable composition with the water phase.

In another embodiment, the oils are selected from the group consisting of glycerides, aliphatic esters, ethers and alkanes, tocopherols, tocopherol esters, esters of salicylic acid and benzoic acid.

In another embodiment the ratio of aromatic/aliphatic oily excipients is between 1:2 and 5:1 by weight. A person skilled in the art after reading the present application would be able to adjust the ratio as desired.

In one embodiment the non-ionic surfactants are present in the self-emulsifiable composition in a total amount from about 5 to 25% by weight.

In yet another embodiment of the invention the self-emulsifable composition of the invention can be used in the formation of an emulsion for topical application. In one embodiment the resulting emulsion is stable. In another embodiment, the oil droplets of the emulsion are of submicron size. In yet another embodiment, the overall size distrubtion of the oil droplets in the emulsion is a narrow sze distribution.

In yet another embodiment, the invention provides an emulsion for topical delivery of salicylates comprising the self-emulsifable composition of the invention as described herein. In one embodiment the emulsion is stable. In one embodiment, the emulsion has a high oil phase content.

In yet another embodiment, the invention provides a process for making the emulsion of the invention comprising combining the self-emulsifiable composition of the invention with a water phase. In one embodiment, the ratio of self-emulsifiable composition to water phase used in the invention is from 2:1 to 1:20. In one embodiment the emulsion has a high oil phase content. In another embodiment the invention provides an emulsion that can be formed by the process of the invention.

In another embodiment the emulsion can be combined with other excipients , such as rheology modifiers to produce a desired rheology. For instance the emulsion can be formed into a cream, lotion or the like.

In yet another embodiment, the invention provides a method or use of the self-emulsifiable composition and emulsions and compositions of the invention in the prevention and/or treatment of pain or as an anti-inflammatory or other uses of salicyaltes.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from reading the detailed description.

### BRIEF DESCRIPTION OF THE FIGURES

FIG. 1 is a graph illustrating the comparative anti-inflammatory action of topical formulations as described in Example 24.

FIG. 2 is a graph illustrating the comparative suppression of carrageenan induced inflammation at maximal edema development as described in Example 24.

### DETAILED DESCRIPTION

Definitions

“ALIPHATIC” as used herein is any open chain organic compound that does not contain aromatic rings.

“AROMATIC COMPOUND” as used herein is any organic compound that contains the molecular structure of which incorporates one or more planar cyclic sets of six carbon atoms that are connected by delocalised electrons numbering the same as if they consisted of alternating single and double covalent bonds.

“SALICYLATE” as used herein is an ester of salicylic acid.

“SALICYLATE ESTERS” as used herein are esters of salicylic acid including, but not limited to, methyl salicylate, ethyl salicylate, ethylhexil salicylate (octyl salicylate), glycol salicylate, and compositions that comprise a salicylate ester, such as wintergreen oil.

“SALICYLIC ACID” as used herein is a 2-Hydroxybenzoic acid.

“EXCIPIENT” as used herein is an ingredient contained in a drug composition that is not an medicinally active compound.

“SURFACTANT” as used herein is a soluble surface acting agent that reduces the surface tension between particulate matter and water.

“ADJUVANT” as used herein is an agent in a composition which modifies the effects of other agents in the composition but has not effect on its own.

“TOPICAL” as used herein is means applied directly to the outer surface of the body, e.g., skin.

“SELF-EMULSIFIABLE” as used herein is a preparation that is capable of forming an emulsion when an aqueous phase is added with minimal input of energy. For example, a self-emsulsifying preparation might form an emulsification upon addition of water combined with slow mechanical stirring. (For instance, as described in U.S. Pat. No. 6,221,919, entitled, “Utilization of ethoxylated fatty acid esters as self-emulsifiable compounds”). Self-emulsifiable compositions are particularly useful when mixtures containing an aqueous phase, generally water, must be prepared without the need for an efficient means of stirring.

“STABLE” as used herein in reference to the stability of a emulsion, formed after dilution of self-emulsifiable composition of the invention, means that state wherein there is no visible phase separation, creaming or precipitation of the emulsion. In one aspect it refers to a period of stability of the emulsion of 3 or more months. In another embodiment it refers to stability of an emulsion of at least 3 months, at room temperature.

“WATER PHASE” as used herein is a continuous phase of an oil-in-water emulsion, usually comprising water; and which might further contain pH adjusting compounds, rheology modifier(s), water-soluble antioxidants, preservatives, colorants, and the like.

“OIL PHASE” as used herein is a discontinuous phase of an oil-in-water emulsion, consisting of oily material(s), wherein other components are dissolved or dispersed: active compound(s), modifiers, lipid-soluble antioxidants, preservatives, fragrances, and the like.

“SUBMICRON” as used herein refers to a size range below 1 micron (1000 nm).

“HIGH PRESSURE HOMOGENIZATION” as used herein is process whereby high pressure (usually 200-2000 bar) is applied to a treated mixture, which causes the mixture to be pumped with high velocity through a narrow channel. Material disruption is then caused by a cavitation process after an abrupt drop in pressure from high pressure described above to atmospheric pressure. The main types of equipment used in high pressure homogenization include Avestin, Gaulin AGV and Microfluidizer homogenizers.

“HIGH SHEAR HOMOGENIZATION” as used herein is a process using equipment which pumps treated mixture with high velocity through a narrow gap, usually in rotor-stator type of mixers. The equipment includes Polytron, Ultra-Turrax, Silverson, OMNI, and colloidal mills of different types.

“ENHANCED ACTIVITY” as used herein, such as in reference to enhanced anti-inflammatory and pain relieving activity, refers to traditional salicylate formulations.

“HIGH CONTENT OIL PHASE” as used in reference to the emulsion obtained from the self-emulsifiable composition of the invention or emulsion composition of the invention refers to 25-60% by weight of oils (including components in the oil phase) in the final emulsion.

“HIGH CONTENT SURFACTANTS AND EMULSIFIERS” as used in reference to the self-emulsifiable composition or emulsion composition of the invention refers to limitation of maximal total content of surfactants and emulsifiers in emulsion, prepared in accordance with the current invention to no more than about 10-15% by weight.

“HIGH CONCENTRATION OF SALICYLATES” as used in this invention in reference to concentration of salicylates in the emulsion, obtained from the self-emulsifiable composition or emulsion composition of the invention means about 10-25% content of salicylate by weight in the final composition.

Abbreviations:

MCT as used herein refers to medium chain triglycerides (mixed capric, caprylic esters of glycerine).
IPM—Isopropyl myristate, isopropyl ester of myristic acid.
Myvacet K-45—acetylated soya oil monoglycerides (Quest International).
Tocophersolan (TPGS)—PEG 1000 ester of tocopheryl succinate (Eastman).
Solutol HS-15—Polyoxyl-15 hydroxystearic acid (BASF).

### DETAILED DESCRIPTION OF THE INVENTION

The present invention relates to a composition for a topical application of salicylates and a method of preparation of salicylate loaded oil-in-water (“O/W”) emulsions. A stable formulation with a high content of oil phase capable of carrying a high concentration of salicylates and showing enhanced efficiency was obtained by dilution of a formerly prepared self-emulsifiable mixture of drug, oil phase and surfactants. The composition can be used for treating pain and inflammation and shows enhanced anti-inflammatory activity.

Salicylates that can be used in the compositions of the invention include but are not limited to Methyl Salicylate, Ethyl Salicylate, Glycol Salicylate, Ethylhexyl Salicylate, Wintergreen oil.

In order to attain superior efficiency in terms of drug loading, physical stability and increased transdermal penetration, both a high content of oil phase and an appropriate size of the emulsion oil droplets are important. During development, it was established that salicylates seriously deteriorate during the emulsification process, providing very coarse unstable O/W emulsions with a strong tendency toward separation. It was also noted that the use of a high content (up to 20-25%) by weight of various ionic surfactants and emulsifiers and different types of glycerides, alkanes, and silicones results in poor emulsions. Furthermore, high-pressure homogenization does not improve the stability of salicylate emulsions.

Surprisingly, it was found that combination of aliphatic and aromatic oily excipients in the oil phase allows successful incorporation of salicylates into emulsion, and the use of a mixture of non-ionic surfactants provides self-emulsifiable properties to the composition.

In one embodiment the aliphatic and aromatic oily excipients are combined in ratio from 1:2 to 5:1 by weight.

Examples of suitable aliphatic oily excipients that are suitable for the invention include but are not necessarily limited to glycerides, aliphatic esters or ethers and alkanes.

Examples of suitable aromatic oily excipients that are suitable for the invention include but are not necesssarily limited to tocopherols, tocopherol esters, esters of salicylic acid or benzoic acid.

Suitable non-ionic surfactants that are suitable for invention include but are not necessarily limited to physiologically acceptable ethoxylated derivatives of fatty acohols, fatty acids, lanolin alcohol, chloseterol, tocopherol, hydroxystearic acid, castor oil, polysorbates, etc.

Moreover, this combination makes it possible to attain a high level of the oil-up to 50-60% w/w in the stable final emulsions and to achieve drug loading in range 10-20%. Unexpectedly, it was also established that combining salicylates, oil phase and surfactants with water phase in certain conditions produces an emulsion with droplets of narrow size distribution and improved stability without the use of any homogenization process and without the use of a protic organic solvents. One aspect of the “certain conditions” used in the invention to form the emulsion, are described below.

In one aspect, the invention is a self-emulsifiable composition which is used in the preparation of an O/W emulsion. The composition is comprised of an oil phase which contains salicylates and a combination of surfactants, in addition to various oils. The components are combined together, slightly heated to liquefy the surfactants, if necessary, and mixed to obtain a homogenous mixture which is the self-emulsifiable composition.

The self-emuslifiable composition (the concentrate) can then be diluted with a water phase to create an oil-in-water(O/W) emulsion. In one embodiment, dilution with the water phase is carried out at temperature of about 60-90° C. The ratio of water phase to oil phase may vary widely, but the best results in terms of physical stability were achieved with 20-60% w/w of oil phase. The resulting O/W emuslion is stable.

In one embodiment, the oil droplets in the emulsion are of submicron size.

After dilution of the self-emusifiable composition with the water phase, the resulting emulsion will be in a lotion form and can be used “as is” as a topical composition. However, in one embodiment, a rheology modifier can be added to the emulsion to obtain a desired thickness for dosage on the skin. In one embodiment, the addition of rheology modifier results in a semi-solid state of formed emulsion. Different types of rheology modifiers can be utilized: Carbopols and other acrylates, polysaccharide gums such as, for example, Xanthan gum, Locust bean gum, Hyaluronic acid, Cellulose derivatives, Starch derivatives, Colloidal Silicon Dioxide, hydrated silicates such as Veegum or Bentonite, Crothix, or the like. Appropriate rheology modifiers are well known in the art, such as those described in [Remington: The Science and Practice of Pharmacy, 20^{th} edition, Lippincott, Williams and Wilkins, Phyladelphia, 2000, pp. 848, 1030-1031; Kibbe, A. H., Handbook of Pharmaceutical Excipients, 3^{rd} Edition, London, 2000] A person skilled in the art would know that the amount of rheology modifier used can be varied depending on the desired resulting thickness of the emulsion composition. In one embodiment, 0.3-1.0% by weight of the rheology modifier is added to the emulsion composition and stirred until the thickener is evenly distributed and hydrated. In one embodiment, the pH of the composition is then adjusted to pH 5.0-7.0 using a suitable pH adjusting agent, e.g., triethanolamine. A person skilled in the art would appreciate that other suitable excipients or adjuvants can be added to compositions as desired, for instance antibacterial preservatives (e.g., Bronopol, Imidazolinylurea), antioxidants, fragrances and color/dye may be added.

The emulsion compositions of the invention can be applied or administered topically to a subject in need or suspected to be in need thereof for the treatment or prevention of pain and/or inflammation. In one embodiment, an effective or therapeutically effective amount of the emulsion is administered. An “effective amount” as used herein means an amount effective, at dosages and for periods of time necessary to achieve the desired results. Administration of a therapeutically effective amount of pharmaceutical compositions of the present invention is defined as an amount effective, at dosages and for periods of time necessary to achieve the desired therapeutic result. For example, an effective or therapeutically effective amount of a substance may vary according to factors such as the disease state, age, sex, and weight of the individual, and the ability of the substance to elicit a desired response in the individual. Dosage regimes may be adjusted to provide the optimum response. For example, several divided doses may be administered daily or the dose may be proportionally reduced as indicated by the exigencies of the therapeutic situation.

The present invention is described in reference to the following Examples, which are set forth to aid in the understanding of the invention, and should not be construed to limit in any way the scope of the invention as defined in the claims which follow thereafter.

### EXAMPLES

### Examples 1-14

### Preparation of Methylsalicylate Cream

1. Emulsion Preparation

A. Oil phase preparation: All components except water were combined while mixing and heated using a water bath (50-60° C.) to obtain a clear transparent solution.

B. Hot water (60-90° C.) was added to oil phase, and a water-in-oil (W/O) emulsion was obtained. That emulsion was mixed using a propeller type mixer for 5-10 minutes. During the following cooling process, the emulsion changed its physical structure, reversing from a W/O emulsion to an O/W emulsion when the temperature decreased below the cloud point of the surfactants in the mixture. Examples 1, 2, and 4-7 result in unstable emulsions, and the formulations in examples 3, 8-14 correspond to stable oil-in-water emulsions.

TABLE 1
Methyl Salicylate Emulsion Compositions

| | Example number: | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| | Percentage, w/w | | | | | | | | | | | | | |
| Oil Phase | | | | | | | | | | | | | | |
| Methyl | 10 | 10 | 10 | 15 | 20 | 15 | 18 | 16 | 16 | 16 | 16 | 16 | 20 | 18 |
| Salycilate | | | | | | | | | | | | | | |
| MCT oil | | 5 | | | 5 | | 5 | 5 | 5 | 5 | 4 | 4 | | 5 |
| L-Menthol | 4 | 4 | 4 | | 4 | | 3 | 4 | 4 | 4 | 6 | 6 | 6 | 6 |
| D,L-Camphor | 4 | 4 | 4 | | 4 | | 5 | 4 | 4 | 4 | 6 | 6 | 6 | 6 |
| IPM | | | | 5 | | 8 | | | | | | | | |
| Myvacet K-45 | 5 | | 5 | | | | | | | | | | 4 | |
| Tocopherol | | 5 | 5 | | | | | 5 | 3 | 2 | 4 | 4 | 4 | 4 |
| acetate | | | | | | | | | | | | | | |
| Lecithin S-80 | 1 | 1 | 1 | | 1 | 1 | 1 | 1 | | 1 | 1 | 1 | 1 | 1 |
| Ethanol | 2 | 2 | 2 | 2 | 2 | 2 | 2 | 2 | 2 | 2 | 2 | 2 | 2 | 2 |
| Tween-80 | 4 | 4 | 4 | | 5 | 4 | 5 | 5 | 4 | 4 | | | | |
| Tocophersolan | 3 | 3 | 3 | 3 | 3 | 2 | 4 | 3 | 3 | 3 | 3.2 | | | |
| Polyethoxylated | | | | 4 | | | | | | | | 4 | | 5 |
| (35) castor oil | | | | | | | | | | | | | | |
| Solutol HS-15 | | | | | | | | | | | 5 | | 3 | |
| Polychol-15 | | | | | | | | | | | | 4 | | |
| Supersat AWS- | | | | | | | | | | | | | 6 | |
| 24 | | | | | | | | | | | | | | |
| Forlan C-24 | | | | | | | | | | | | | | 5 |
| Water phase % | 67 | 62 | 62 | 71 | 56 | 68 | 57 | 55 | 59 | 59 | 52.8 | 53 | 48 | 48 |
| Total: | 100% | 100% | 100% | 100% | 100% | 100% | 100% | 100% | 100% | 100% | 100% | 100% | 100% | 100% |
| Stability of | − | − | + | − | − | − | − | +++ | ++ | + | +++ | +++ | ++ | +++ |
| emulsion* | | | | | | | | | | | | | | |

*Used symbols of emulsion stability description:
‘−’ - very unstable, visible phase separation immediately after preparation or in 0.5-1 hour
‘+’ - unstable, visible phase separation in 24 hours storage at room temperature
‘++’ - moderately stable, signs of separation after 2-4 weeks storage at room temperature
‘+++’ - very stable —no signs of phase separation after 6 months and more

2. Cream Preparation

Selected stable emulsions were mixed with 0.3-1.0% by weight of a thickener (e.g., Carbopol 934P,971P, 1342P, 974P, Ultrez 10, Ultrez 21), and stirred until the thickener was evenly distributed and hydrated. After hydration, pH was adjusted to 5.0-7.0 using triethanolamine, and the resultant product was mixed thoroughly providing a smooth cream. If desired, antibacterial preservatives (e.g., Bronopol, Imidazolinylurea), antioxidants, fragrances and color/dye may be added.

### Example 15

TABLE 2
Ethyl Salicylate composition

| | |
|---|---|
| Oil Phase | |
| Ethyl salicylate | 12% |
| Myvacet 9-45K | 4% |
| L-Menthol | 6% |
| D,L-Camphor | 6% |
| Tocopherol acetate | 4% |
| Lecithin S-80 | 1% |
| Ethanol | 1% |
| Solutol HS-15 | 4% |
| Tocophersolan | 4% |
| Water phase | 58% |
| Total: | 100% |
| Stability of emulsion | +++ |

### Example 16

TABLE 3
Octylsalicylate (Ethylhexyl salicylate) composition

| | |
|---|---|
| Oil Phase | |
| Octyl salicylate | 10% |
| MCT oil | 6% |
| L-Menthol | 6% |
| D,L-Camphor | 6% |
| Tocopherol acetate | 2% |
| Lecithin S-80 | 1% |
| Ethanol | 1% |
| Cremophor RH-40 | 5% |
| Tocophersolan | 4% |
| Water phase | 59% |
| Total: | 100% |
| Stability of emulsion | ++ |

The preparation process for Examples 15 and 16 was similar to that for Examples 1-14

### Examples 17-21

TABLE 4
Glycol salicylate compositions

| | 17 | 18 | 19 | 20 | 21 |
|---|---|---|---|---|---|
| Oil Phase | | | | | |
| Glycol salicylate | 10.0 | 10.0 | 10.0 | 10.0 | 15.0 |
| MCT oil | 12.0 | | | | |
| Myvacet 9-45K | | 12.0 | 12.8 | 12.0 | 10.0 |
| L-Menthol | 2.7 | 2.7 | 2.4 | 2.5 | 3.0 |
| D,L-Camphor | 2.5 | 2.5 | 2.4 | 2.5 | 3.0 |
| Tocopherol acetate | 4.0 | 4.0 | 3.2 | 4.0 | 6.0 |
| Lecithin S-80 | 0.8 | 0.8 | 0.5 | 0.8 | 1.0 |
| Ethanol | 1.0 | 1.0 | 0.5 | | 1.0 |
| Cremophor EL | 5.0 | 5.0 | 4.8 | 5.0 | 5.0 |
| Tocophersolan | 2.0 | 2.0 | 1.6 | 2.0 | 2.0 |
| Water phase | 60 | 60 | 61.8 | 61.2 | 54.0 |
| Total: | 100% | 100% | 100% | 100% | 100% |
| Stability of emulsion | − | +++ | ++ | + | +++ |

All components of the oil phase were combined and heated to 65-75° C., accompanied by slow mixing until homogenous clear solution was obtained. Water phase was added and the formed emulsion was stirred for an additional 20 minutes using a propeller type mixer. After cooling, the required amount of rheology modifier (Carbopol) was added, the system was mixed for an additional 30-60 minutes, and the pH was adjusted with triethanolamine. Formed cream was stirred using a planetary mixer for 30 minutes.

### Example 22

TABLE 5
Wintergreen oil cream composition

| | |
|---|---|
| Wintergreen oil | 15% |
| MCT oil | 5.5% |
| L-Menthol | 4% |
| D,L-Camphor | 4% |
| Tocopherol acetate | 5% |
| Lecithin S-80 | 1% |
| Ethyl alcohol | 1% |
| Arnica extract 1:10 (Flavex) | 2.6% |
| Marygold CO2 extract (Flavex) | 0.10% |
| Sage CO2 extract (Flavex) | 0.10% |
| Chamomile CO2 extract (Flavex) | 0.10% |
| Tween-80 | 5% |
| Tocophersolan (Eastman) | 3% |
| Hypericum perforatum CO2 extract | 0.10% |
| Labrasol (Gattefosse) | 1% |
| EDTA | 0.05% |
| Carbopol 934 P | 1% |
| Triethanolamine | 0.40% |
| Bronopol | 0.10% |
| Glycerin | 2.4% |
| Water | 48.55% |
| Total | 100.00% |

Cream with Methylsalicylate of natural origin (wintergreen oil, Methyl Salicylate content >90%) with added natural herbal extracts was prepared similarly to examples 1-14.

TABLE 6
Median particle size of salicylate ester emulsions, prepared from self-emulsifiable
compositions (Data obtained before addition of rheology modifier)

| | Example number: | | | | | | | | | | | |
|---|---|---|---|---|---|---|---|---|---|---|---|---|
| | 1 | 8 | 9 | 11 | 14 | 15 | 16 | 17 | 18 | 19 | 20 | 22 |
| | Shimadzu SALD-2001 results, nm | | | | | | | | | | | |
| D50 | N/A | 390 | 720 | 330 | 560 | 420 | 760 | N/A | 365 | 450 | 1600 | 900 |
| Stability of emulsion | − | +++ | ++ | +++ | +++ | +++ | ++ | − | +++ | ++ | + | ++ |

### Example 23

Association of Salicylate with the Oil Phase

Association of the salicylate with the oil phase of prepared emulsion was estimated using ultrafiltration technique. 400 mcl of prepared emulsion was placed into centrifugal ultrafiltration system (“Ultrafree-MC” Sigma-Aldrich, regenerated cellulose membrane, molecular weight cutoff 30,000 Dalton) and centrifugated at 1000 g for 2 hours. Clear solution, passed membrane, was analysed for content of Salicylates using HPLC method. Results are presented in Table 7.

TABLE 7

| | | Association |
|---|---|---|
| Example Number | Salicylate ester | with oil phase |
| Example 5 | Methyl | N/A (emulsion broken) |
| Example 10 | Methyl | 93% |
| Example 11 | Methyl | 95% |
| Example 15 | Ethyl | 98% |
| Example 16 | Ethylhexyl (octyl) | >99% |
| Example 19 | Glycol | 74% |
| Example 21 | Glycol | 85% |

### Example 24

Enhanced Activity of Methylsalicylate in Proposed Vehicle

Anti-inflammatory activity evaluation (gamma-carrageenan induced paw edema model in rats)

Male Wistar rats (150-200 g) were housed in air-conditioned room at 22±1° C. and constant humidity. Standard diet and water available ad libitum. For injection of carrageenan solution rats were anesthetized with Halothane. Carraggenan solution was injected intraplantarly into right hind paw. Carrageenan was prepared as 1% solution in sterile saline 24 h before an experiment by continuous stirring with heating at 60° C. on magnetic stirrer (0.5-1 hour). This protocol prevented the appearance of behavioral signs of pain during and after the injections.

Each animal is marked at Tibia-Calcaneus joint of the hind paw using waterproof marker. For topical delivery 100 microliters of formulation were applied immediately after Carrageenan insult onto plantar surface.

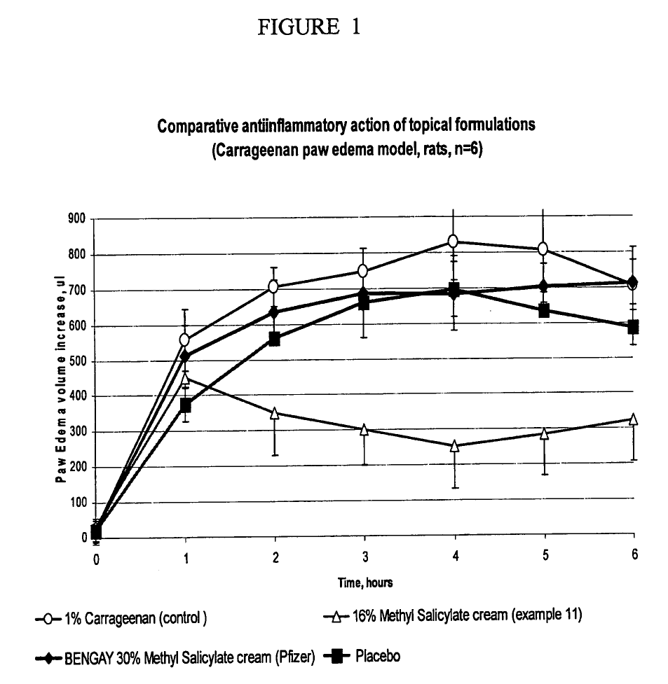

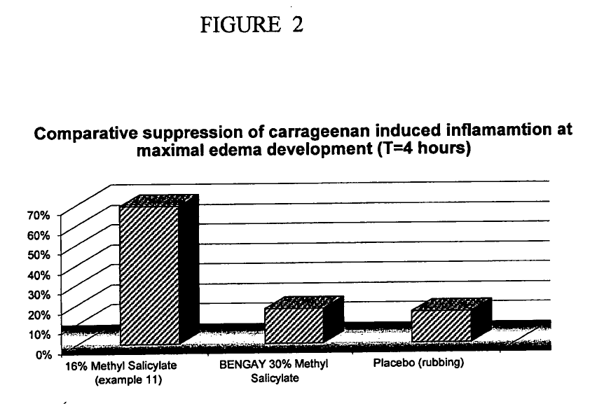

Intraplantar injections were made with a disposable syringe (0.5 ml) and a 30-gauge hypodermic needle in volume of 100 mcl. The volume of the carrageenan-injected and contralateral saline-injected paw is measured using a plethysmometer Model 7141 (Ugo Basile, Varese, Italy). The plethysmometer was calibrated and tested according to manufacturer's Manual. Foot volumes were measured immediately before the injection of carrageenan at different intervals thereafter ( 1 h, 2 h, 3 h, 4 h, 5 h and 6 h). The paw is dipped into a cylinder filled with water (contain 3 ml/l of wetting compound and 0.5 g/l of Sodium chloride), exactly up to the reference mark on the paw. The difference (ml) in volume between the right paw (carrageenan-injected) and left paw (saline-injected) was calculated (“delta”) and used as estimate of edema. Results are presented in FIG. 1 and FIG. 2.

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims. It should be noted the application is intended to encompass obvious chemical equivalents of the components of the invention as described herein, which are equivalents that produce the same or equivalent desired result for a particular feature of the invention, e.g. self-emulsifiable composition, emulsions, compositions, processes of making same and uses thereof. In this regard, the term “about” as used herein is intended to encompass such obvious chemical equivalents.

All publications, patents, and patent applications are herein incorporated by reference in their entireties, to the same extent as if each individual publication, patent, or patent application was specifically and individually indicated to be incorporated by reference in its entirety.

What is claimed is:

1. A self-emulsifiable composition that can be used in the formulation of a topical salicylate ester emulsion composition comprising
(a) a salicylate ester;
(b) oils comprising a mixture of aromatic and aliphatic oily excipients in a ratio of 1:2 to 5:1 by weight;
(c) non-ionic surfactants.

2. A self-emulsifiable composition of claim 1 comprising 20-50% by weight of salicylate ester.

3. A self-emulsifiable composition of claim 1 wherein the salicylate ester is selected from the group consisting of Methyl salicylate, Ethyl salicylate, Glycol salicylate, and Ethylhexyl salicylate.

4. A self-emulsifiable composition of claim 1 comprising 5-25% by weight of oils.

5. A self-emulsifiable composition of claim 4 wherein the oils are selected from the group consisting of glycerides, aliphatic esters, ethers and alkanes, tocopherols, tocopherol esters, esters of salicylic acid and benzoic acid.

6. A self-emulsifiable composition of claim 1 comprising 5-25% by weight of non-ionic surfactants.

7. A self-emulsifiable composition as set forth in claim 1, wherein the composition forms a stable oil-in-water emulsion upon dilution with water phase, wherein the composition comprises 10-60% of the emulsion.

8. An emulsion comprising the self-emulsifiable composition as set forth in claim 6 wherein the salicylate ester is predominantly associated with the oil phase of the emulsion after dilution with water phase.

9. A composition as set forth in claim 6 wherein the emulsion additionally comprises one or more rheology modifiers producing a semisolid composition suitable for topical application.

10. A composition for the preparation of the salicylate ester loaded topical formulation comprising salicylate ester, oil phase, oil phase modifiers, and surfactant or mixture of surfactants.

11. A composition as set forth in claim 10 wherein the oil phase comprises a mixture of aliphatic and aromatic components.

12. A composition as set forth in claim 11 wherein the aliphatic component of said oil phase is selected from the group consisting of glycerides, aliphatic esters, ethers and alkanes.

13. A composition as set forth in claim 12 wherein the aromatic component of said oil phase is selected from the group consisting of tocopherols, tocopherol esters, esters of salicylic acid and benzoic acid.

14. A composition as set forth in claim 11 wherein the aliphatic and aromatic components of the oil phase are present in a ratio between 1:2 and 5:1 by weight.

15. A composition as set forth in claim 10 where the oil phase modifiers are polar compounds, soluble in the oil phase.

16. The modifiers as set forth in claim 15 wherein the polar compounds are aliphatic alcohols or ketones, or a combination thereof.

17. A composition as set forth in claim 16 wherein the polar compounds are camphor or menthol, or a combination thereof.

18. A composition as set forth in claim 17 wherein the camphor and menthol mixture constitutes between 5% and 80% of the oil phase by weight.

19. A composition as set forth in claim 16 wherein menthol and camphor are present in a ratio between 1:2 and 2:1 by weight.

20. A process for the preparation of a stable submicron emulsion of salicylates wherein the self-emulsifiable composition of claim 1 is diluted with water phase to a ratio from 2:1 to 1:20.

21. A process for the preparation of a stable submicron emulsion of salicylates by dilution of a self-emulsifiable composition followed by agitation, wherein the process provides spontaneous formation of a submicron emulsion and does not involve the application of high energy/ high force emulsification, such as high pressure homogenization, high shear homogenization, sonication, or passage through microporous membranes.

22. The composition of claim 7 with enhanced anti-inflammatory and pain relieving activity.

23. A method of treating an inflammatory and/or pain condition comprising administering an effective amount of the composition of claim 1 to a patient in need thereof.
